(19) United States
(12) Patent Application Publication
(10) Pub. No.: US 2010/0099732 A1
(43) Pub. Date: Apr. 22, 2010

(54) MEDICAMENTS USEFUL AS POTASSIUM CHANNEL MODULATORS

(75) Inventors: Antonio Nardi, Ballerup (DK); Morten Grunnet, Kobenhavn O (DK); Palle Christophersen, Ballerup (DK); David Spencer Jones, Smorum (DK); Elsebet Østergaard Nielsen, Kobenhavn K (DK); Dorte Strøbaek, Farum (DK); Lars Siim Madsen, Soro (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/532,264

(22) PCT Filed: Mar. 14, 2008
(86) PCT No.: PCT/EP2008/053052

§ 371 Date: Nov. 24, 2009

(30) Foreign Application Priority Data
Mar. 22, 2007 (DK) PA 2007 00444

(51) Int. Cl. A61K 31/40

(52) U.S. Cl. 514/419

(57) ABSTRACT

This invention relates to pharmaceutical compositions comprising phenylcarbamoyl-methyl-indole derivatives that are found to be potent modulators of potassium channels and, as such, are valuable candidates for the treatment of diseases or disorders as diverse as those which are responsive to the modulation of potassium channels.

### TECHNICAL FIELD

This invention relates to pharmaceutical compositions comprising phenylcarbamoyl-methyl-indole derivatives that are found to be potent modulators of potassium channels and, as such, are valuable candidates for the treatment of diseases or disorders as diverse as those which are responsive to the modulation of potassium channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation.

Dysfunction of potassium channels, as well as other ion channels, generates loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain, and cancer.

Among the large number of potassium channel types, the large-conductance calcium-activated potassium channel subtype is an obvious site for pharmacological intervention and for the development of new potassium channel modulators. Their physiological role has been especially studied in the nervous system, where they are key regulators of neuronal excitability and of neurotransmitter release, and in smooth muscle, where they are crucial in modulating the tone of vascular, broncho-tracheal, urethral, uterine or gastro-intestinal musculature.

Given these implications, small agents with BK-opening properties could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions, anxiety and pain. As far as the cardiovascular system is concerned, the physiological function of these ion channels represents a fundamental steady state mechanism, modulating vessel depolarisation, vasoconstriction and increases of intravascular pressure, and the development of selective activators of BK channels is seen as a potential pharmacotherapy of vascular diseases, including hypertension, erectile dysfunction, coronary diseases and vascular complications associated with diabetes or hypercholesterolemia.

Phenylcarbamoyl-methyl-indole derivatives have been described by Ali et al. [Ali M I, Abdel-Fattah A M, Hussain S M, El-Reedy A M: Reaction of 2-carboxy-1-methylindole-3-acetic acid anhydride with amines and with S-methylisothiosemicarbazide; Journal of Heterocyclic Chemistry 1982 19 (5) 993-996] and are commercially available from e.g. Aurora Screening Library. However, any biological activity has never been reported.

### SUMMARY OF THE INVENTION

It is an object of the invention to provide novel pharmaceutical compositions comprising a therapeutically effective amount of a phenylcarbamoyl-methyl-indole derivative of Formula I

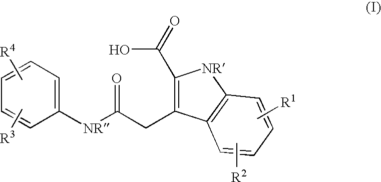

wherein,

R′ and R″, independently of each other, represent hydrogen or alkyl; and

R¹, R², R³ and R⁴, independently of each other, represent hydrogen, alkyl, cycloalkyl, halo, trifluoromethyl, trifluoromethoxy, cyano, nitro, hydroxy, alkoxy, thio-alkoxy, phenyl or phenoxy;

or an enantiomer or a mixture of its enantiomers, or a pharmaceutically-acceptable addition salt thereof, or a prodrug thereof, together with one or more adjuvants, excipients, carriers and/or diluents.

In another aspect the invention provides a method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the phenylcarbamoyl-methyl-indole derivative for use according to the invention.

Other objects of the invention will be apparent to the person skilled in the art from the following detailed description and examples.

### DETAILED DISCLOSURE OF THE INVENTION

In its first aspect the invention provides novel pharmaceutical compositions comprising a phenylcarbamoyl-methyl-indole derivative, or an enantiomer thereof or a mixture of its enantiomers, or a pharmaceutically-acceptable addition salt thereof, or a prodrug thereof, together with one or more adjuvants, excipients, carriers and/or diluents.

The phenylcarbamoyl-methyl-indole derivative for use according to the invention may be characterised by Formula I

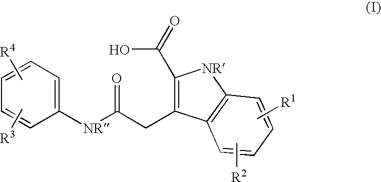

wherein,

R′ and R″, independently of each other, represent hydrogen or alkyl; and

R¹, R², R³ and R⁴, independently of each other, represent hydrogen, alkyl, cycloalkyl, halo, trifluoromethyl, trifluoromethoxy, cyano, nitro, hydroxy, alkoxy, thio-alkoxy, phenyl or phenoxy.

In a preferred embodiment the phenylcarbamoyl-methyl-indole derivative for use according to the invention is a compound of Formula I, wherein R′ and R″, independently of each other, represent hydrogen or alkyl.

In a more preferred embodiment one of R′ and R″ represents hydrogen; and the other of R′ and R″ represents alkyl.

In an even more preferred embodiment both of R′ and R″ represent hydrogen.

In another more preferred embodiment both of R′ and R″ represent alkyl.

In another preferred embodiment the phenylcarbamoyl-methyl-indole derivative for use according to the invention is a compound of Formula I, wherein R¹, R², R³ and R⁴, independently of each other, represent hydrogen, alkyl, cycloalkyl, halo, trifluoromethyl, trifluoromethoxy, cyano, nitro, hydroxy, alkoxy, thio-alkoxy, phenyl or phenoxy.

In a more preferred embodiment one of R¹ and R², and one of R³ and R⁴ represents hydrogen; and the other of R¹ and R², and the other of R³ and R⁴ represent, independently of each other, represent alkyl, cycloalkyl, halo, trifluoromethyl, trifluoromethoxy, cyano, nitro, hydroxy, alkoxy, thio-alkoxy, phenyl or phenoxy.

In an even more preferred embodiment one of R¹ and R², and one of R³ and R⁴ represents hydrogen; and the other of R¹ and R², and the other of R³ and R⁴ represent, independently of each other, represent halo, trifluoromethyl, cyano, nitro, alkoxy, thio-alkoxy or phenoxy. In a still more preferred embodiment one of R¹ and R², and one of R³ and R⁴ represents hydrogen; and the other of R¹ and R², and the other of R³ and R⁴ represent, independently of each other, represent halo, trifluoromethyl, cyano, thio-alkoxy or phenoxy.

In a yet more preferred embodiment one of R¹ and R², and one of R³ and R⁴ represents hydrogen; and the other of R¹ and R², and the other of R³ and R⁴ represent, independently of each other, represent fluoro, chloro, trifluoromethyl, cyano, thio-methoxy or phenoxy.

In a most preferred embodiment the phenylcarbamoyl-methyl-indole derivative for use according to the invention is
5-Chloro-3-[(4-fluoro-phenylcarbamoyl)-methyl]-1H-indole-2-carboxylic acid;
5-Chloro-3-[(3-chloro-phenylcarbamoyl)-methyl]-1H-indole-2-carboxylic acid;
5-Chloro-3-[(3-cyano-phenylcarbamoyl)-methyl]-1H-indole-2-carboxylic acid;
5-Chloro-3-[(4-trifluoromethyl-phenylcarbamoyl)-methyl]-1H-indole-2-carboxylic acid;
5-Chloro-3-[(4-phenoxy-phenylcarbamoyl)-methyl]-1H-indole-2-carboxylic acid;
5-Chloro-3-[(3-methylsulfanyl-phenylcarbamoyl)-methyl]-1H-indole-2-carboxylic acid;
5-Chloro-3-[(3-trifluoromethyl-phenylcarbamoyl)-methyl]-1H-indole-2-carboxylic acid;
5-Chloro-3-[(4-chloro-3-methoxy-phenylcarbamoyl)-methyl]-1H-indole-2-carboxylic acid; or
5-Chloro-3-[(2-chloro-4-fluoro-phenylcarbamoyl)-methyl]-1H-indole-2-carboxylic acid;

or an enantiomer thereof or a mixture of its enantiomers, or a pharmaceutically-acceptable addition salt thereof, or a prodrug thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention an alkoxy group designates an “alkyl-O—” group, wherein alkyl is as defined above. Examples of preferred alkoxy groups of the invention include methoxy and ethoxy.

In the context of this invention an thioalkoxy group designates an “alkyl-S—” group, wherein alkyl is as defined above. Likewise thioalkoxy-alkoxy, alkoxy-thioalkoxy, and thioalkoxy-thioalkoxy designates a thioalkoxy group as defined above, attached to another thioalkoxy group, or to an alkoxy group as defined above.

### Pharmaceutically Acceptable Salts

The phenylcarbamoyl-methyl-indole derivatives for use according to the invention may be provided in any form suitable for the intended administration. Suitable forms include pharmaceutically (i.e. physiologically) acceptable salts, and pre- or prodrug forms of the phenylcarbamoyl-methyl-indole derivative.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a phenylcarbamoyl-methyl-indole derivative for use according to the invention include, without limitation, the sodium, the potassium, the calcium, the magnesium, the lithium, and the ammonium salt, and the like, of a phenylcarbamoyl-methyl-indole derivative containing an anionic group. Such cationic salts may be formed by procedures well known and described in the art.

### Methods of Preparation

The compounds according to the invention may be easily prepared by conventional methods described in the literature, e.g. by Ali et al. [Ali M I, Abdel-Fattah A M, Hussain S M, El-Reedy A M: Reaction of 2-carboxy-1-methylindole-3-acetic acid anhydride with amines and with S-methylisothiosemicarbazide; Journal of Heterocyclic Chemistry 1982 19 (5) 993-996], and will typically involve the reaction between a suitable 4,9-dihydro-pyrano[3,4-b]indole-1,3-diones and an amine.

### Biological Activity

The phenylcarbamoyl-methyl-indole derivatives for use according to the invention have been found to possess potassium channel modulating activity as measured by standard electrophysiological methods. Due to their activity at the potassium channels, the phenylcarbamoyl-methyl-indole derivatives for use according to the invention are considered useful for the treatment of a wide range of diseases and conditions.

In a special embodiment, the phenylcarbamoyl-methyl-indole derivatives for use according to the invention are considered useful for the treatment, prevention or alleviation of a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjogren's syndrome, xerostomia, arrhythmia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.

In a more preferred embodiment, the phenylcarbamoyl-methyl-indole derivatives for use according to the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the phenylcarbamoyl-methyl-indole derivatives for use according to the invention are considered useful for the treatment, prevention or alleviation of psychosis, schizophrenia, bipolar disorder, depression, epilepsy, Parkinson's disease or pain.

In a third more preferred embodiment, the phenylcarbamoyl-methyl-indole derivatives for use according to the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the phenylcarbamoyl-methyl-indole derivatives for use according to the invention are considered useful for the treatment, prevention or alleviation of cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy, cardiomyopathy or a genetic disease.

In a fifth more preferred embodiment, the phenylcarbamoyl-methyl-indole derivatives for use according to the invention are considered useful for the treatment, prevention or alleviation of cardiac ischemia, ischemic heart disease, hypertrophic heart, cardiomyopathy or failing heart.

In a sixth more preferred embodiment, the phenylcarbamoyl-methyl-indole derivatives for use according to the invention are considered useful for the treatment, prevention or alleviation of cardiac arrhythmia, atrial fibrillation and/or ventricular tachyarrhythmia.

In a seventh more preferred embodiment, the phenylcarbamoyl-methyl-indole derivatives for use according to the invention are considered useful for the treatment, prevention or alleviation of schizophrenia, depression or Parkinson's disease.

In an eighth more preferred embodiment, the phenylcarbamoyl-methyl-indole derivatives for use according to the invention are considered useful for the treatment, prevention or alleviation of a sexual dysfunction, incl. male sexual dysfunction and female sexual dysfunction, and incl. male erectile dysfunction.

In an even more preferred embodiment the phenylcarbamoyl-methyl-indole derivatives for use according to the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the phenylcarbamoyl-methyl-indole derivatives for use according to the invention are used in a combination therapy together with sildenafil, tadalafil, vardenafil or calcium dobesilate.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred phenylcarbamoyl-methyl-indole derivatives for use according to the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of a phenylcarbamoyl-methyl-indole derivative for use according to the invention.

While a phenylcarbamoyl-methyl-indole derivative for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the phenylcarbamoyl-methyl-indole derivative for use according to the invention together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method of treatment, prevention or alleviation of a disease, disorder or condition of a living animal body, including a human, which disorder, disease or condition is responsive to activation of a potassium channel, which method comprises the step of administering to such a living animal body in need thereof, a therapeutically effective amount a compound capable of activating the potassium channel, or a pharmaceutically-acceptable addition salt thereof.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWING

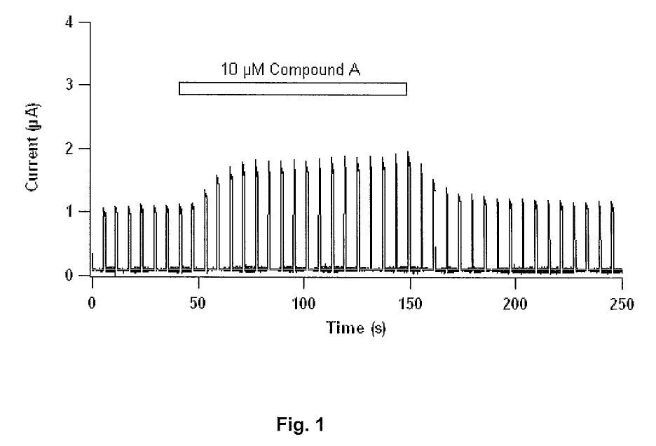

The present invention is further illustrated by reference to the accompanying drawing, in which FIG. 1 shows the BK channel opening activity [current (μA) vs. time (s)] of a phenylcarbamoyl-methyl-indole derivative for use according to the invention, i.e. 5-Chloro-3-[(3-chloro-phenylcarbamoyl)-methyl]-1H-indole-2-carboxylic acid (herein designated Compound A) determined by a standard electrophysiological method using BK channels heterologously expressed in Xenopus laevis oocytes.

### EXAMPLES

The invention is further illustrated with reference to the following examples, which are not intended to be in any way limiting to the scope of the invention as claimed.

### Example 1

### Biological Activity

In this example the BK channel opening activity of a phenylcarbamoyl-methyl-indole derivative for use according to the invention, i.e. 5-Chloro-3-[(3-chloro-phenylcarbamoyl)-methyl]-1H-indole-2-carboxylic acid (herein designated Compound A), is determined using BK channels heterologously expressed in Xenopus laevis oocytes.

The electrical current through the BK channel is measured conventional two-electrode voltage clamp. BK current is activated by repeated step protocols. In brief, this protocol goes from a resting membrane potential of −40 mV lasting for 5 s to a depolarised step to +20 mV lasting for 1 s. The protocol was repeated continuously.

Having reached stable current level, Compound A (10 μM), was added. A marked increase in the current activated by depolarisation could be observed. The BK current activity returned to baseline after approximately 30-60 seconds of wash. In summary BK current was increased by 92±23% in the presence of 10 μM of compound A (n=3, SD value). The results are presented in FIG. 1.

1. A pharmaceutical composition comprising a therapeutically effective amount of a phenylcarbamoyl-methyl-indole derivative of Formula I 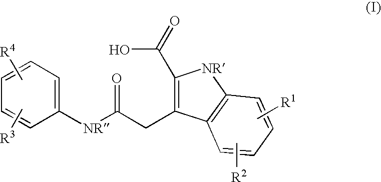
wherein,
R′ and R″, independently of each other, represent hydrogen or alkyl; and
R¹, R², R³ and R⁴, independently of each other, represent hydrogen, alkyl, cycloalkyl, halo, trifluoromethyl, trifluoromethoxy, cyano, nitro, hydroxy, alkoxy, thio-alkoxy, phenyl or phenoxy;
or an enantiomer thereof or a mixture of its enantiomers, or a pharmaceutically-acceptable addition salt thereof, together with one or more adjuvants, excipients, carriers and/or diluents.

2. The pharmaceutical composition of claim 1, wherein R′ and R″, independently of each other, represent hydrogen or alkyl.

3. The pharmaceutical composition of claim 1, wherein R¹, R², R³ and R⁴, independently of each other, represent hydrogen, alkyl, cycloalkyl, halo, trifluoromethyl, trifluoromethoxy, cyano, nitro, hydroxy, alkoxy, thio-alkoxy, phenyl or phenoxy.

4. The pharmaceutical composition of claim 1, wherein the phenylcarbamoyl-methyl-indole derivative is
5-Chloro-3-[(4-fluoro-phenylcarbamoyl)-methyl]-1H-indole-2-carboxylic acid;
5-Chloro-3-[(3-chloro-phenylcarbamoyl)-methyl]-1H-indole-2-carboxylic acid;
5-Chloro-3-[(3-cyano-phenylcarbamoyl)-methyl]-1H-indole-2-carboxylic acid;
5-Chloro-3-[(4-trifluoromethyl-phenylcarbamoyl)-methyl]-1H-indole-2-carboxylic acid;
5-Chloro-3-[(4-phenoxy-phenylcarbamoyl)-methyl]-1H-indole-2-carboxylic acid;
5-Chloro-3-[(3-methylsulfanyl-phenylcarbamoyl)-methyl]-1H-indole-2-carboxylic acid;
5-Chloro-3-[(3-trifluoromethyl-phenylcarbamoyl)-methyl]-1H-indole-2-carboxylic acid;
5-Chloro-3-[(4-chloro-3-methoxy-phenylcarbamoyl)-methyl]-1H-indole-2-carboxylic acid; or
5-Chloro-3-[(2-chloro-4-fluoro-phenylcarbamoyl)-methyl]-1H-indole-2-carboxylic acid;
or an enantiomer thereof or a mixture of its enantiomers, or a pharmaceutically-acceptable addition salt thereof.

5. The pharmaceutical composition of according claim 1, for the treatment, prevention or alleviation of a disease or a disorder or a condition of a mammal, including a human, which disease, disorder or condition is responsive to modulation of potassium channels.

6. The pharmaceutical composition of claim 5, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjogren's syndrome, xerostomia, arrhythmia, cardiovascular disorders, hypertension, myotonic dystrophy, myotonic muscle dystrophia, spasticity, xerostomi, diabetes Type II, hyperinsulinemia, premature labour, cancer, brain tumors, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.

7. A method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of a phenylcarbamoyl-methyl-indole derivative, or an enantiomer thereof or a mixture of its enantiomers, or a pharmaceutically-acceptable addition salt thereof, as described in claim 1.
